(19) United States
(12) Patent Application Publication
(10) Pub. No.: US 2008/0159604 A1
(43) Pub. Date: Jul. 3, 2008

(54) Method and system for imaging to identify vascularization

(76) Inventors: Allan Wang, Irvine, CA (US); Igor Ternovskiy, Rancho Palos Verdes, CA (US)

Correspondence Address: LAWRENCE S. COHEN, Suite 1220, 10960 Wilshire Blvd., Los Angeles, CA, 90024, US

(21) Appl. No.: 11/323,877

(22) Filed: Dec. 30, 2005

(51) Int. Cl. G06K 9/00; A61B 5/05

(52) U.S. Cl. 382/128; 600/410; 600/407

(57) ABSTRACT

Apparatus and method for determining an extent of vascularization in which a digitaldigitalized representation of blood vessels in a selected area is generated; one or more statistical quantative measures for the blood vessels in the selected area are calculated; and the one or more statistical quantative measures are compared to corresponding statistical standards to determine an extent of vascularization. The statistical quantative measures may include the density of branch points and the density of end points in a skeleton representing the blood vessels and a fractal dimension for the skeleton.

### GOVERNMENT LICENSE RIGHTS

The U.S. Government has a paid-up license in this invention and the right in limited circumstances to require the patent owner to license others on reasonable terms as provided for by the terms of Contract No. DAMD17-01-C-0020 awarded by the Department of Defense.

### BACKGROUND

1. Field

This disclosure relates to a method and system for analysis of images for vascularization. More particularly, the present disclosure describes modeling used to classify data acquired from imaging analysis to identify and analyze vascularization for an angiogenesis determination.

2. Description of Related Art

Vascularization is a process by which body tissue becomes vascular and develops capillaries veins and arteries. A particular case of vascularization, Angiogenesis, i.e., the formation of new blood vessels, is a critical component in many physiological and pathological processes, such as in the healing of wounds, bone fractures, ulcers, and in diseases, such as cancer, renal pathy, rheumatoid arthritis, cardiological recovery and evaluation and smoking related pathological changes. Prior art recognizes that angiogenesis may indicate the presence of a problem in a patient or may be encouraged to remedy a problem in a patient. Prior art related to angiogenesis includes the following U.S. patents and U.S. patent application publications:

| U.S. Pat. No. | Title | Issue Date |
|---|---|---|
| U.S. Pat. No. 5,941,832 | Method and apparatus for detection of cancerous and | Aug. 24, 1999 |
| | precancerous conditions in a breast | |
| U.S. Pat. No. 6,211,157 | Protein mixtures to induce therapeutic angiogenesis | Apr. 3, 2001 |
| U.S. Pat. No. 6,385,474 | Method and apparatus for high-resolution detection and | May 7, 2002 |
| | characterization of medical pathologies | |
| U.S. Pat. No. 6,389,305 | Method and apparatus for detection of cancerous and | May 14, 2002 |
| | precancerous conditions in a breast | |
| U.S. Pat. No. 6,455,311 | Fabrication of vascularized tissue | Sep. 24, 2002 |
| U.S. Pat. No. 6,673,908 | Tumor necrosis factor receptor 2 | Jan. 6, 2004 |
| U.S. Pat. No. 6,728,567 | Method and apparatus for high-resolution detection and | Apr. 27, 2004 |
| | characterization of medical pathologies | |
| U.S. Pat. No. 6,836,557 | Method and system for assessment of biomarkers by | Dec. 28, 2004 |
| | measurement of response to stimulus | |

| Pat. App. Pub No. | Title | Publication Date |
|---|---|---|
| US 20020040004 | Method of promoting natural bypass | Apr. 4, 2002 |
| US 20020065466 | Method and apparatus for high-resolution detection | May 30, 2002 |
| | and characterization of medical pathologies | |
| US 20020182241 | Tissue engineering of three-dimensional | Dec. 5, 2002 |
| | vascularized using microfabricated polymer | |
| | assembly technology | |
| US 20030003575 | Fabrication of vascularized tissue using | Jan. 2, 2003 |
| | microfabricated two-dimensional molds | |
| US 20030100824 | Architecture tool and methods of use | May 29, 2003 |
| US 20030186334 | KTS-disintegrins | Oct. 2, 2003 |
| US 20040066955 | Method and system for assessment of biomarkers | Apr. 8, 2004 |
| | by measurement of response to stimulus | |
| US 20040147830 | Method and system for use of biomarkers in | Jul. 29, 2004 |
| | diagnostic imaging | |
| US 20040252870 | System and method for three-dimensional image | Dec. 16, 2004 |
| | rendering and analysis | |
| US 20040253365 | Architecture tool and methods of use | Dec. 16, 2004 |
| US 20050143312 | Compositions and methods for promoting | Jun. 30, 2005 |
| | myocardial and peripheral angiogenesis | |
| US 20050175540 | High contrast optoacoustical imaging using | Aug. 11, 2005 |
| | nonoparticles | |

Imaging devices used for medical purposes, such as laser Doppler devices, magnetic resonance imaging units, or ultrasonic equipment, may include image analysis software, but such software typically does not address either vascularization or angiogenesis analysis. Well-known imaging analysis programs, such as NIH ImageJ, ImageProPlus, and Halcon, are not typically used for vascularization or angiogenesis analysis, but may be adapted for use in such analysis. However, the functions provided in such programs may have significant shortcomings in their use for such analysis. Therefore, there exists a need in the art for a system and method for analyzing images to identify vascularization, particularly in the identification of angiogenesis.

### SUMMARY

Embodiments of the present invention are based on the recognition that the development of a blood vessel network necessary for sustaining certain physiological and/or pathological processes, such as for tumor growth, can be identified by the statisticalquantative and statistical examination of an image or images morphology, or data representative of an image or images, of an area in the vicinity of the blood vessel network. The statisticalquantative and statistical examination provides one or more statistical quantative measures related to the vascularization depicted in the image or images. Embodiments according to the present invention provide for rapid evaluation of blood supply changes, but allow for such evaluation to be performed without rigorous registration of multiple images with one another. Embodiments of the present invention also allow for the image analysis to be based on only a relatively few parameters, which, in turn, provides for an attractive input regimen for neural network evaluation. The base line for blood supply growth analysis is obtained by analyzing the status of tiny blood vessels in order to provide a reference assessment.

An embodiment of the present invention is method for determining an extent of vascularization comprising: generating a digitaldigitalized representation of blood vessels in a selected area; calculating one or more statistical quantative measures for the blood vessels in the selected area based on the digitaldigitalized representation of the blood vessels; and comparing the one or more statistical quantative measures to corresponding statistical standards to determine an extent of vascularization. The digitaldigitalized representation of blood vessels may be a binarized image of the vessels presented as a skeleton of the blood vessel network. The one or more statistical quantative measures may be the density of branch points and the density of end points in the skeleton and the fractal dimension of the skeleton. The extent of vascularization may indicate the presence or absence of angiogenesis.

Another embodiment of the present invention is a system for determining the extent of vascularization in a subject where the system has: an input device operable to receive one or more images of blood vessels in the subject; an output device; and a processor programmed to: generate a digitaldigitalized representation of the blood vessels in a selected area based on the one or more images of blood vessels in the subject; calculate one or more statistical quantative measures for the blood vessels in the selected area based on the digitaldigitalized representation of the blood vessels; compare the one or more statistical quantative measures to corresponding statistical standards to determine an extent of vascularization; and send results based on the comparison of the one or more statistical quantative measures to corresponding statistical standards to the output device. The digitaldigitalized representation of blood vessels may be a binarized image of the vessels presented as a skeleton of the blood vessel network. The one or more statistical quantative measures may be the density of branch points and the density of end points in the skeleton and the fractal dimension of the skeleton. The extent of vascularization may indicate the presence or absence of angiogenesis.

Another embodiment of the present invention is a method for determining the presence of angiogenesis comprising: receiving an image of a blood vessel network; extracting an area of interest in the received image; converting the area of interest in the received image to a plurality of line segments, wherein only blood vessels in the area of interest having a thickness in excess of a selected thickness are converted to line segments; connecting neighboring line segments and eliminating gaps between line segments to generate a skeleton representing the blood vessels; calculating a fractal dimension for the skeleton; comparing the fractal dimension to a fractal dimension threshold value; and generating a result indicating a presence of angiogenesis if the fractal dimension exceeds the fractal dimension threshold value. The method may further comprise: calculating a density of branch points in the skeleton; comparing the density of branch points to a branch point threshold value; and generating a result indicating a presence of angiogenesis if the density of branch points exceeds the branch point threshold value or calculating a density of end points in the skeleton; comparing the density of end points to an end point threshold value; and generating a result indicating a presence of angiogenesis if the density of end points exceeds the end point threshold value. A Graphical User Interface may be used to display the images and/or select portions of the images for processing. The Graphical User Interface may also be used to display the angiogenesis determination results.

### BRIEF DESCRIPTION OF THE DRAWINGS

FIG. 1 shows a typical image displaying a blood vessel network.

FIG. 2 shows detected lines overlaid on the image of FIG. 1 using one set of line detection parameters.

FIG. 2 shows detected lines overlaid on the image of FIG. 1 using a different set of line detection parameters.

FIG. 4 shows a relatively large blood vessel.

FIG. 5 shows detected lines overlaid on the image of FIG. 4, where it can be seen that the walls of the thick blood vessels are detected as lines.

FIG. 6 shows another large blood vessel.

FIG. 7 shows detected lines overlaid on the image of FIG. 6, where it can be seen that one of the walls of the thick blood vessel is detected as a line, instead of the vessel center.

FIGS. 8A-8C show the improvements that may be obtained in line detection using techniques according to an embodiment of the present invention.

FIGS. 9A-9C show the improvements that may be obtained in line detection using techniques according to an embodiment of the present invention.

FIG. 10 shows the Quotient Index obtained from the analysis of CAM image data from several different days.

FIGS. 11A-11C show plots of branch points, end points, and fractal dimension versus the day of CAM images.

FIGS. 12A-12C show the distribution of results for the three parameters for the CAM images for different days.

FIGS. 13A-13C show the application of the regression statistics against the CAM image data.

FIG. 14 shows a flow diagram of an embodiment of the present invention for identifying and analyzing angiogenesis.

FIGS. 15A and 15B show a GUI provided by an embodiment of the present invention.

FIG. 16A shows an original brain MRA and FIG. 16B shows the same MRA after performing line detection in accordance with embodiments of the present invention.

FIG. 17 shows an exemplary GUI display for the analysis of a brain MRA image.

### DETAILED DESCRIPTION

Embodiments of the present invention base the determination of the presence of angiogenesis on one or more of the three following factors for a blood vessel network: number of branch points, number of end points, and a fractal dimension for the network. These factors are calculated based on an image showing the blood vessel network. The image may be provided by, for example, a Magnetic Resonance Angiogram (MRA). However, to ensure that these factors are correctly calculated based on the image, embodiments of the present invention base the calculations on a set of lines. i.e., a skeleton, extracted from the image that represents the blood vessel network. Further, these factors may also be used in a more general determination of the extent of vascularization depicted in an image.

As indicated above, embodiments of the present invention utilize an image (or images) showing a blood vessel network to perform the angiogenesis analysis. However, before performing line detection in the image, it is preferable that certain operations be performed on the image to enhance the image to improve the quality of the line detection. The imaging operations that may be performed include:

1. Converting the image to grayscale, so that each pixel has a range of gray intensity (blood vessel images, such as MRA images, are typically color images);

2. Inverting the image, so that brighter (higher intensity) areas correspond to blood vessels;

3. Equalizing the contrast across the image to compensate for variations in lighting;

4. Maximizing the contrast of the image; and

5. Performing an opening operation (erosion of the white areas followed by dilation by the same amount) to remove small areas of noise.

Contrast equalization (step 3 above) may be accomplished by taking a local minimum in each of a set of regions in the image to establish a local lighting level, interpolating the regional data to produce an estimate of lighting intensity at each pixel in the image (i.e., to form a background intensity image), and subtracting the background intensity image from the data image.

In one embodiment of the present invention, an entire image provided by imaging machinery may be processed and analyzed in accordance with the techniques described herein. In another embodiment, a user may select a portion or portions of the original image for processing and analysis. For example, a doctor, when viewing an MRA image of a large area, may select an area of interest within the large area image for processing and analysis in accordance with the techniques described herein. In still another embodiment, an automated process may be used to divide a large area image into several smaller sized images, where each of the smaller sized images are processed and analyzed.

Additional processes or other processes may be used to manipulate the original image to improve the line detection process described below. The preferred result of these processes is to binarize the image, that is, produce a converted image where pixel values of 1 represent blood vessels and pixel values of 0 represent the lack of blood vessels.

As indicated above, the blood vessel network shown in an image can be defined by performing line detection on the image. Line detection is similar to edge detection in that the image is searched for specific spatial changes. However, because lines are distinct from edges, edge filters alone are typically not adequate for this task. Several different line detectors are known in the art. For example, the Halcon commercial image processing software provides a line detection filter. The Halcon manual discloses:
“The line extraction is done by using partial derivatives of a Gaussian smoothing kernel to determine the parameters of a quadratic polynomial in x and y for each point of the image. The parameters of the polynomial are used to calculate the line direction for each pixel. Pixels which exhibit a local maximum in the second directional derivative perpendicular to the line direction are marked as line points. The line points found in this manner are then linked to contours”

In a preferred embodiment, line detection was performed by immediately accepting line points that had a second derivative larger than a user specified threshold. Points that had a second derivative smaller than a second user specified threshold were rejected.

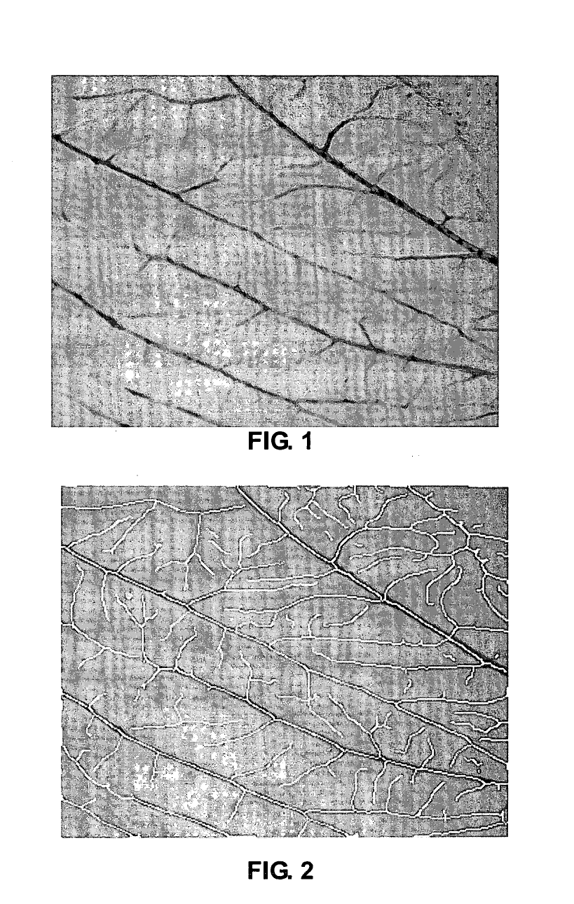

FIG. 1 shows a typical image displaying a blood vessel network. FIG. 2 shows detected lines overlaid on the image of FIG. 1 using a sigma of 1.5 and a threshold for acceptance of 2.0 and a threshold for rejection of 0.5. FIG. 3 depicts the same image using different parameter values for accepting and rejecting line elements with a threshold for acceptance of 0.5 and a threshold for rejection of 0.0 (sigma still 1.5). As can be seen from FIGS. 2 and 3, lowering the thresholds does result in finding more vessels, but, also, more points that were probably not vessels were found.

The thresholds used to generate the detected lines shown in FIGS. 2 and 3 were determined by using several different thresholds and then determining which threshold seemed to provide the best results, i.e., detected lines that correctly overlaid the blood vessels depicted in the image. The process of determining thresholds for line detection may also be implemented using a neural network learning process, where a large sample size of images may be analyzed for the determination of optimum line detection thresholds to be used. The sample size of images may also take into account different situations, e.g., images of breast tissue versus images of brain tissue, to allow for the determination of different line detection thresholds for these different situations.

In an exemplary embodiment, original images of 1280×1024 pixels were reduced by a factor of 4 to speed image processing. Such a reduction does not appear to affect the accuracy of the results, so other embodiments may use additional reduction factors.

Once lines are detected within an image, the detected line information may be stored within a data structure that preferably stores the position and width of the detected lines to subpixel accuracy. For example, if the Halcon software is used for line detection, the detected lines may be stored in a particular data structure within Halcon.

Once the data structure is generated, additional processing is preferably performed to calculate the following values for each detected line segment: length, width, standard deviation of the width, and area. The length, width and area values are preferably calculated as numbers of pixels.

Additional processing is also preferably performed to further characterize the detected lines by calculating the number of endpoints and junctions. A skeletonization algorithm is preferably used to provide for the ready calculation of these parameters. Filtering of the data before performing these calculations may or may not be done. Small noise segments that are incorrectly identified as lines may be removed by removing line segments smaller than a few pixels, perhaps on the order of 5 pixels or fewer.

Several potential artifacts may influence system accuracy. The potential artifacts include the interference of thick vessel walls and the detection of fibrous structures as vessels. Also, for images that comprise images of the brain or skull area, blood vessels located on the boundary of the skull may skew system accuracy. Therefore, preferred embodiments of the present invention implement techniques to address these artifacts.

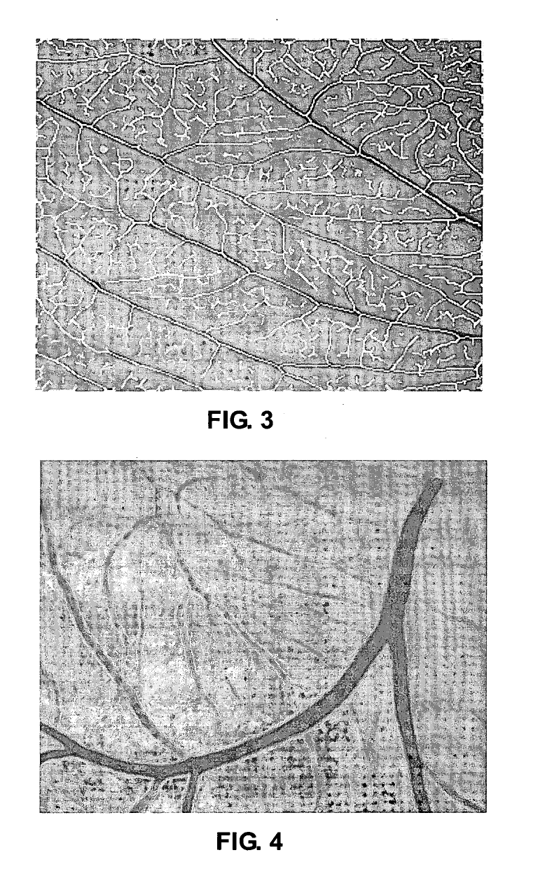

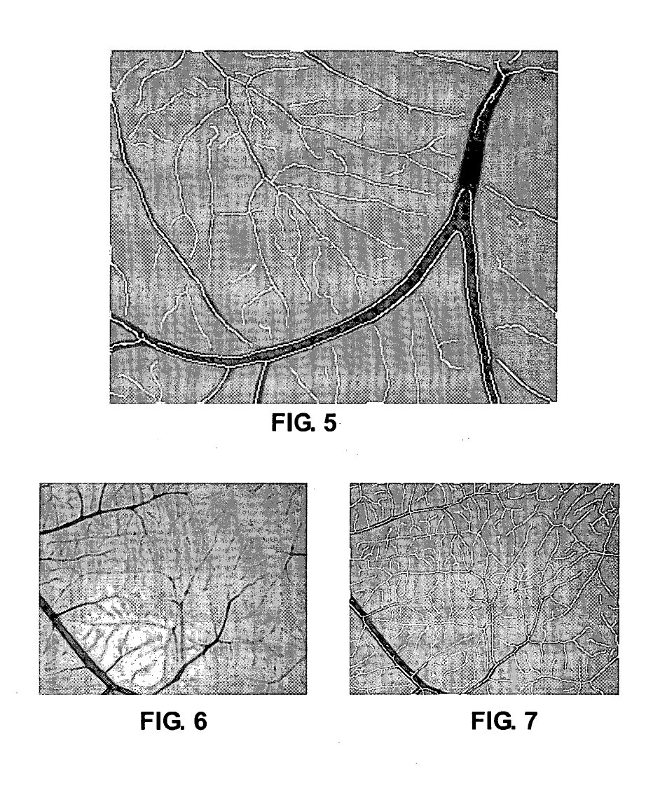

Large blood vessels can be artificially recognized as two smaller blood vessels because of the thickness of the vessel. The line detection algorithm described above may detect the vessel walls as lines instead of the vessel centers because: (1) the vessel wall is distinctly different colored and looks like a line, or (2) the blood vessel is large with respect to the parameters used for an edge detector, or (3) the uneven thickness of the two sides of vessel walls. A combination of these effects is also possible. FIG. 4 shows a relatively large blood vessel and FIG. 5 shows detected lines overlaid on the image of FIG. 4, where it can be seen that the walls of the thick blood vessels are detected as lines, rather than the blood vessels themselves. FIG. 6 shows another large blood vessel and FIG. 7 shows detected lines overlaid on the image of FIG. 7, where it can be seen that one of the walls of the thick blood vessel is detected as a line, instead of the vessel center.

One approach for removing the artifacts is to downsample the image into several different smaller scales. Lines can then be detected on several scales and the best information on each scale can be combined into a final result. The small lines will be degraded in the smaller image, and therefore, will not be found. The thick lines will also be made smaller, and will match more appropriately with the size of the lines expected by the line detector. This is more computationally efficient than increasing the size of the filter. Joining information from different scales can be complicated, however, because there is conflicting information depending on the thickness, position, and orientation of a line at a particular scale.

Another approach for removing the artifacts is to perform a segmentation of the image. The segmentation of the large white regions is fairly accurate, but the segmentation of the smaller vessels is problematic. The image can be searched for parallel lines (ribbons), where elongated segments with parallel lines on either side are identified as vessels. This information is consistent with line detectors that work on the smaller scales where the segmentation results begin to fail. This approach may provide the ability to find the larger thicker vessels where the line detection algorithm fails.

A preferred algorithm for removing artifacts induced by vessel walls first requires defining two lines that are “near” to each other. Then, an average distance between all points on the lines is computed. This can be performed by integrating the distances measured between all points of each line. For most cases, the area between two line elements can be computed using basic geometry. The area can be divided by the length of the longest line element to create an estimate for the average distance between the two lines. If, for example, a threshold of 3 pixels is set for this quantity (i.e., the distances between the line), it means that thin lines that are more than 3 pixels (on average) away from the thick line need to be removed.

Two parallel lines that have the same orientation, but are lined up side by side, are the types of lines that need to be removed. Determining the lines that have similar orientation and are side by side, may be accomplished by defining the relative angle between the two lines. To take the entire line segment information into account, an “X” shape can be drawn between the endpoints of the two line segments, and the angle of the intersection computed. If this angle is small, then the lines are assumed to be nearby, and side-by-side. However, an intersecting “X” shape cannot always be drawn from the endpoints of the two lines. In fact, this may occur only about 3-4% of the time. In these cases, an angle may not be estimated, but, instead, the decision on removing lines may be based solely on the area and distance between the lines.

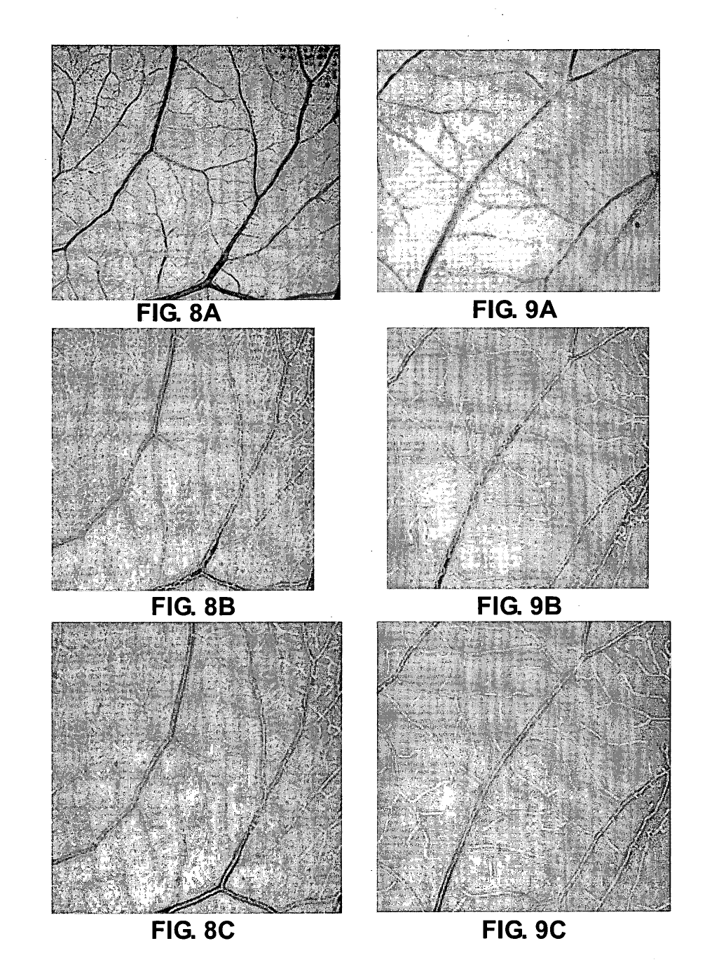

FIGS. 8A-8C and 9A-9C show the improvements that may be obtained in line detection by using the artifact removal techniques described above. FIG. 8A shows an original image with several thick blood vessels. FIG. 8B shows the image of 8A after line detection. FIG. 9A shows another original image with several vessels of varying thickness and FIG. 9B shows the image of 9A after line detection. Both FIGS. 8B and 9B show that using the techniques described above, lines along vessel walls are eliminated and that the lines are typically located in the center of the vessels rather than close to the walls. However, in both FIGS. 8B and 9B, noises. i.e., extraneous short line segments still appear. FIGS. 8C and 9C show the additional improvement that may be obtained by preprocessing the image before performing line detection. Specifically, FIGS. 8C and 9C are the line detection results obtained when the original images are first preprocessed using the grayscaling and contrast enhancing techniques described above before performing line detection. As can be seen in FIGS. 8C and 9C, almost none of the unwanted small line segments appear where there are no vessels. Further, the accuracy of line detection on thick blood vessels is also improved.

Some embodiments of the present invention provide a method for distinguishing angiogeneic from non-angiogeneic vascular images. This method allows angiogenesis to be identified from any single image of vessels by comparing the image to an angiogenesis imaging library. This method is much more convenient and objective than analysis conducted by an experienced cytologist using a manual method. The method is based on the analysis of data from several blood vessel parameters: the number of branch points, the number of terminating (end) points, fractal dimension, and the total lengths of vessels with sections of various widths of 0-1 pixels, 1-2 pixels, and so on, up to widths greater than 8 pixels. StatisticalQuantative and statistical analysis of these measurements indicates that a linear relationship exists between the levels of angiogenesis and the quantification of the first three parameters (branch points, end points, and fractal dimensions). These three parameters are described in additional detail below.

A conventional quantification of angiogenesis may be accomplished by manually counting the number of blood vessel branch points viewed within an image. The branch points comprise the points where one vessel splits into two. The end points comprise the ends of each vessel in the image, if shown in the image. As discussed above, the skeletonization of the detected lines in an image may assist in automatically counting the number of branch points and end points or other processing methods may be used to count the number of branch points and end points. Conventional software such as the Halcon software may also provide functions for counting the branch points and end points in detected lines.

Fractals are loosely thought of as patterns exhibiting self-similarity, fractional dimension, or set uncountability. A primary feature of fractals is the property that an increase in magnification yields similar-looking patterns. Blood vessels exhibit this complex feature. As the angiogenesis process becomes more developed, and more vessels grow, the value of the fractal dimension increases. Embodiments of the present invention may use fractal dimension for determination of vascularization or angiogenesis.

A preferred method, according to the present invention, for computing the fractal dimension for a processed image is the “box counting algorithm,” which is well known in the art. In accordance with an embodiment of the present invention, after the blood vessels in an image are converted to lines, the images of the lines are placed in a 300 pixel by 300 pixel window. This window size is arbitrary and can be optimized as necessary, so alternative embodiments may use different window sizes. Next, a variety of observing boxes are created (in the window) in various decreasing sizes. In one embodiment, the lengths (L) of the observing boxes created are 300, 27, 14, 9, 7, and 5 pixels, but alternative embodiments may again use different sizes for the observing boxes. The image is filled with these observing boxes, and then the number of observing boxes (total=N) that contain blood vessel pixels are calculated. Once these values are calculated, a log-log plot of N as a function of L is generated. The negative of the slope value of this plot is the measured fractal dimension (FD) as shown by the equation below.

FD=log(N)/log(1/(L/300))=−log(N)/log(L/300)

Other embodiments of the present invention may use other algorithms for calculating the fractal dimension of the blood vessels in an image as represented by detected lines, e.g., such as the well-known “ruler algorithm” originally developed by Lewis Fry Richardson. Essentially, the fractal dimension provides a measure of how complicated a blood vessel image is. In accordance with embodiments of the present invention, this measure provides a statistical quantative measure of the vascularization shown in an image, which may then be used for an angiogenesis determination.

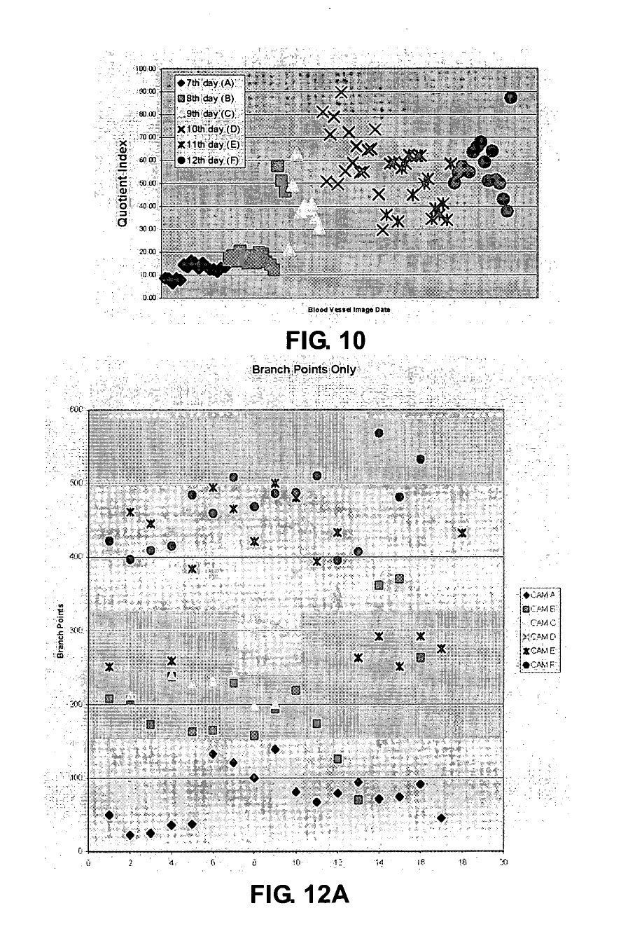

The parameters described above were used to characterize images obtained from chick embryo chorioallantoic membrane (CAM) vessel images from a 7^{th} day to a 12^{th} day (Group A to Group F). To establish a baseline, all the CAM data from day 7 was averaged (A). The parameters obtained from each image at days 7 through 12 were divided by the average value of (A). All the quotients were then summed for each image and plotted in an angiogenesis chart shown in FIG. 10. This method of calculating the Quotient Index appears to demonstrate a distinct separation of the different levels of angiogenesis among 7th to 12th day CAM. There appears to be a clear discontinuity for the data between Group B and C at around the quotient index of 25.

Table 1 below shows a sample of angiogenesis analysis for the CAM images based on the parameters discussed above:

TABLE 1

| Parameters | | | Total Lengths of Vessels with Various Widths | | | | | | | | |
|---|---|---|---|---|---|---|---|---|---|---|---|
| | | Fract | 0-1 | 1-2 | 2-3 | 3-4 | 4-5 | 5-6 | 6-7 | 7-8 | |
| Branch Points | End Points | Dim. | Pixels | Pixels | Pixels | Pixels | Pixels | Pixels | Pixels | Pixels | Above 8 Pixels |

Average Data of Any Group:

| | | | | | | | | | | | |
|---|---|---|---|---|---|---|---|---|---|---|---|
| 74.412 | 184 | 1.7945 | 123.24 | 16.381 | 38.78 | 257.33 | 2926.5 | 2714.4 | 666.81 | 245.09 | 171.06 |

Data From a Single Group:

| | | | | | | | | | | | |
|---|---|---|---|---|---|---|---|---|---|---|---|
| 407 | 612 | 1.9375 | 255.87 | 160.88 | 344.43 | 3106.6 | 5479.6 | 3685.4 | 905.17 | 613.74 | 8.0476 |

Ratio of Data from Single Group to Average Data of Any Group:

| | | | | | | | | | | | |
|---|---|---|---|---|---|---|---|---|---|---|---|
| 5.4696 | 3.3261 | 1.0797 | 2.0762 | 9.8207 | 8.8816 | 12.072 | 1.8705 | 1.3577 | 1.3575 | 2.5041 | 0.0470 |

Embodiments of the present invention provide for analysis of parameters from images that correlate with the amount of angiogenesis. As discussed above, these parameters include: 1) the density of the branch points, 2) the density of the end points, and 3) the fractal dimension. The analysis also includes the thickness distribution of blood vessels by determining the total length of all blood vessels between zero and one pixels wide, the total length of all blood vessels between one and two pixels wide, and so on, through the total length of all blood vessels between eight and nine pixels wide (parameters 4 through 12). Inspection of plots of angiogenesis and these parameters for CAM images shows that most of the parameters show a strong correlation with angiogenesis during various days of embryo development, and that the correlation is rather linear and nonnegative. Hence, embodiments of the present invention employ a linear model for characterizing angiogenesis.

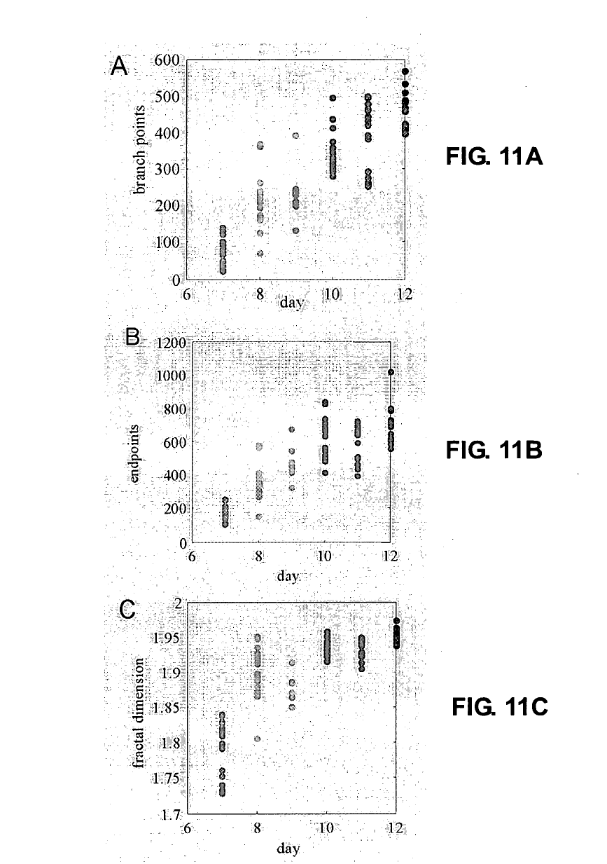

Statistical analysis of the CAM data set used to generate FIG. 10 showed that the first eight parameters of Table 1 were highly significant (p<0.0001, t>5.6, df=91, in double sided t-test) while the last four parameters were not significant (p>0.1, t<1.5, df=91). The data indicated that blood vessels with diameters of over 5 pixels were existing vessels, and that the length distribution of those large vessels was not related to angiogenesis. Using the statisticalquantative and statistical analysis of the CAM images, the normalized correlation coefficients of the 12 parameters were found to be: 0.8824, 0.8320, 0.8009, 0.5092, 0.6972, 0.6371, 0.6521, 0.6802, −0.1099, −0.0844, 0.1648, and −0.0948, respectively. Among the eight highly significant parameters, the first three (density of branch points, the density of end points, and the fractal dimension) had the strongest correlation. FIGS. 11A-11C show plots of these values versus the day of the CAM images, which illustrates the correlation of these values with the number of days. Specifically, FIG. 11A shows the branch points, FIG. 11B shows the endpoints, and FIG. 11C shows the fractal dimension.

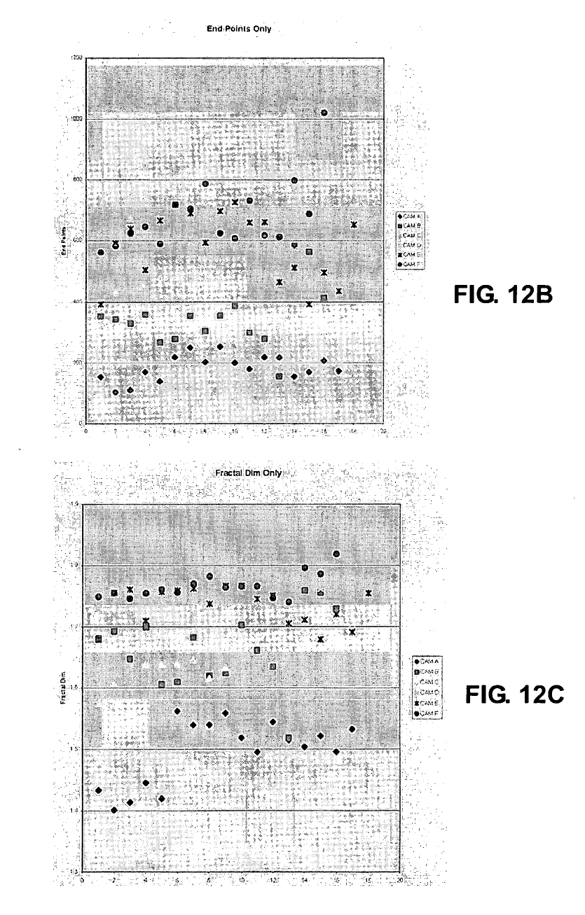

With regard to the CAM images, manual counting results and cytological examination of the those images showed that the 9^{th} day CAM was recognized as the starting day of CAM angiogenesis. FIGS. 12A-12C show the distribution of results for the three parameters for the CAM images for different days (CAM A=7 days, CAM B=8 days, CAM C=9 days, CAM D=10 days, CAM E=11 days, CAM F=12 days). FIG. 12A shows that the angiographies from the CAM D group and beyond have high counts of branch points (over 300 branch points per image) and are clearly separated from the groups of CAM A through CAM C. FIG. 12B shows that high counts of end points (over 400) are seen for the angiographies of CAM C and beyond. The separation between angiogeneic and non-angiogeneic angiographies, however, is not as distinct as for the branch point testing. StatisticalQuantative and statistical analysis shows that end point counting is basically parallel to branch point counting. This suggests that the counting of end points is not required if counting of the branch points is performed. Counting fewer parameters may allow for testing to be performed more rapidly. FIG. 12C shows good correlation of the angiogenesis data from the CAM C group and beyond (FDs of 1.6 and higher). The results clearly indicate that angiogenesis started with CAM C, which is the 9th day of CAM. These results are similar to those from the branch point counting data, but show a better separation of angiogenesis and non-angiogenesis. Hence, based on this analysis, FD testing may be considered to be the best approach for distinguishing between angiogeneic and non-angiogeneic angiographies, while branch point counting may be considered to be the second best approach. Embodiments of the present invention may use any combination of branch point testing, end point testing, and fractal dimension counting.

As indicated above, embodiments of the present invention may employ a linear model to correlate a particular parameter to angiogenesis. However, statisticalquantative and statistical analysis of the fractal dimension data from the CAM images indicated that an exponential model may also be used for the correlation of that data with angiogenesis. The linear regression model is based on a “least squares” method to calculate a straight line to fit the data and the exponential model calculates an exponential curve to fit the data. Considering a line in the form of y=m*x+b and an exponential curve in the form of y=b*m^{x}, Table 2 below shows the regression statistics obtained from the CAM images discussed above for end point, branch point, and fractal dimension data.

TABLE 2

| | | Value from | Value from | Value from | Value from |
|---|---|---|---|---|---|
| | | Branch Point | End Point | Fractal Dim. | Fractal Dim. |
| Regression | | Linear | Linear | Linear | Exponential |
| Statistic | Description | Regression | Regression | Regression | Regression |
| m | Coefficient | 97.31429 | 128.611 | 0.058211 | 1.039 |
| b | Constant | −592.41 | −657.798 | 1.0094 | 1.0815 |
| se | The standard error value for the | 5.268 | 7.745 | 0.003649 | 0.0025 |
| | coefficient m | | | | |
| se_{b} | The standard error value for the | 51.15334 | 75.236 | 0.0354 | 0.0242 |
| | constant b | | | | |
| R₂ | Coefficient of determination. | 0.79 | 0.75 | 0.7365 | 0.722 |
| | Compares estimated and actual | | | | |
| | y-values | | | | |
| seᵥ | The standard error for the y | 89.565 | 131.733 | 0.0620 | 0.0424 |
| | estimate | | | | |

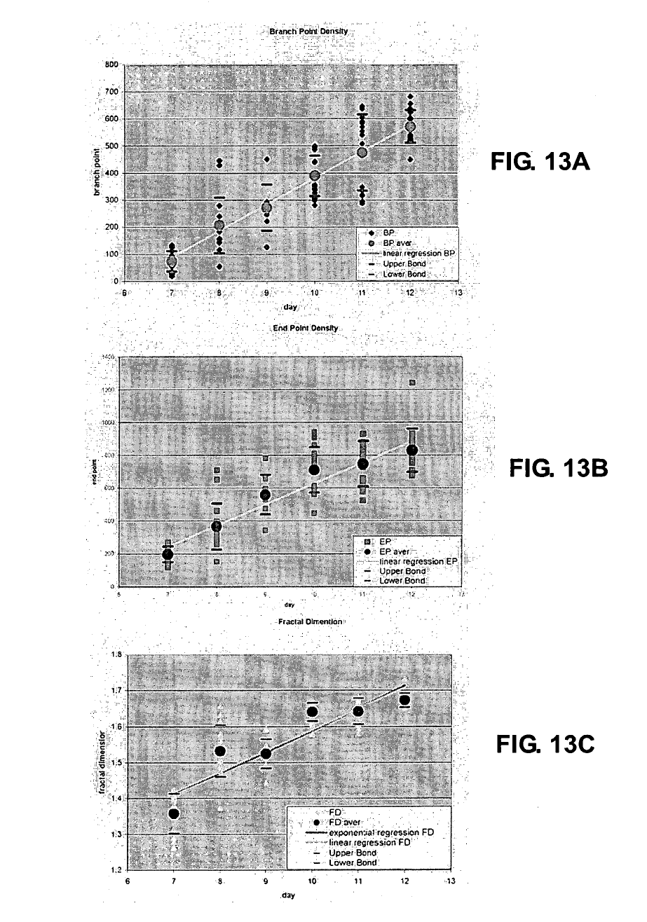

FIGS. 13A-13C show the application of the regression statistics against the CAM image data. FIG. 13A shows the branch point data, the average point, and the resultant point from the linear regression for the CAM image data for different days. FIG. 13B shows the end point data and FIG. 13C shows the fractal dimension data. As shown in FIG. 13C, the exponential and linear regression is nearly the same, so preferred embodiments of the present invention use a linear model for fractal dimension data. FIGS. 13A-13C also demonstrate that the three criteria of branch point, end point and fractal dimension provide a significant diagnosis tool to diagnose angiogenesis.

FIGS. 13A-13C also show that the branch point data, the end point data, and the fractal dimension data may also be used to provide some statistical quantative measure of the vascularization depicted in an image or a series of images. FIGS. 13A-13C show that these data may change over time, so that the statistics related to the data provide an indication of the change in vascularization over time. The rate of change of vascularization may be used to provide insight into other conditions related to vascularization besides angiogenesis.

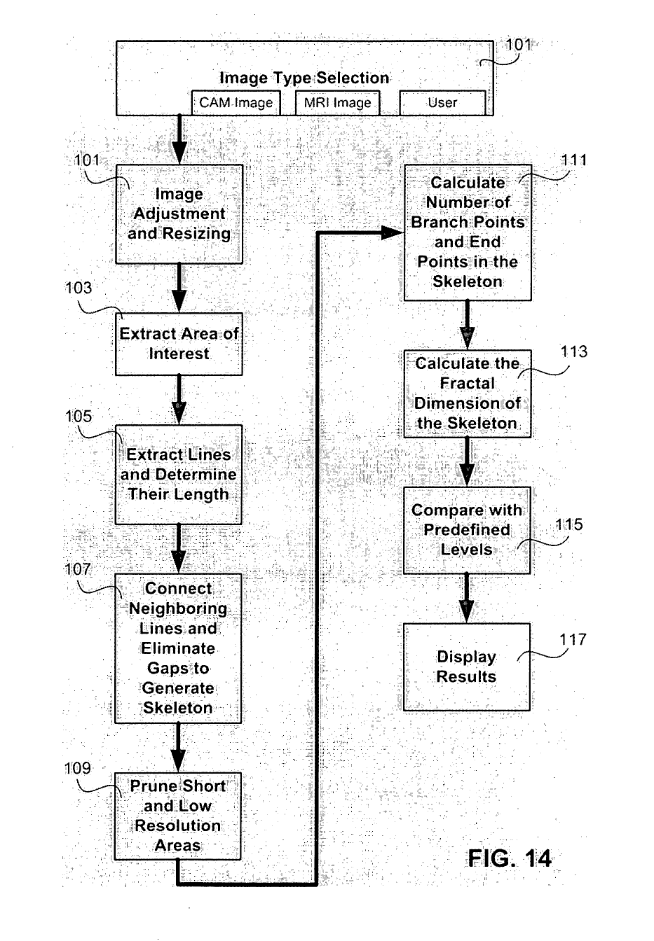

FIG. 14 shows a flow diagram of an embodiment of the present invention for identifying and analyzing angiogenesis. Block 101 shows the selection of an image. The image may be automatically provided or may be selected by a user. The image preferably comprises an image of a blood vessel network, such as provided in a CAM image, an MRI image, or other styles of images that depict blood vessel networks.

After the image is selected, Block 103 shows that the image may be adjusted and/or resized. Adjustments to the image include the operations described above related to grayscaling, contrast enhancement, etc. As discussed above, these operations may be performed to improve the quality of line detection of the blood vessel network.

After any image adjustment is performed, Block 105 shows that line detection related to the depicted blood vessels is performed. As discussed above, line detection algorithms known in the art may be used. For example, the line detection algorithm may comprise the following steps:

1. Use partial derivatives of a Gaussian smoothing kernel to determine the parameters of a quadratic polynomial in x and y for each point of the image.

2. Calculate the line direction for each pixel using the parameters.

3. Mark as line points the pixels that exhibit a local maximum in the second directional derivative perpendicular to the line.

4. Link line points to contours by accepting line points that have a second derivative larger than the (user specified) threshold.

5. Reject points that have a second derivative smaller than a second (user specified) threshold (Sigma values).

Further, the line detection may also include those techniques discussed above used to address thick blood vessels. After the lines are detected, the lengths of the detected lines are determined. The result of the processing depicted in Block 105 is a binarized image, where a pixel value of one represents the presence of a blood vessel and a zero represents the absence of a blood vessel. However, the additional processing described below in regard to Blocks 107 and 109 is preferably also performed to improve the binary representation of the blood vessel network.

Block 107 shows the conversion of the detected lines to a skeleton by connecting neighboring lines and eliminating gaps. Skeletonization algorithms known in the art may be used to accomplish this or other methods may be used to provide the desired representation of the blood vessel network as a skeleton.

Block 109 shows the preferred step of eliminating short lines and eliminating low resolution areas in the skeleton, i.e., pruning the skeleton of these items. As discussed above, short lines and low resolution areas may be indicative of noise rather than actual blood vessels.

Block 111 shows the calculation of branch points and end points in the skeleton. Techniques and algorithms known in the art may be used to calculate the branch points and end points. Branch points comprise those points where a line splits into two and the end points comprise the ends of the lines.

Block 113 shows the calculation of the fractal dimension for the skeleton. The fractal dimension is calculated as discussed above, where the image containing the skeleton is divided by a number of lines into squares with various dimensions (e.g., 1, 5, 10, and 20 lines). The number of squares that contain any lines or branch points are then counted. The fractal dimension is calculated as the slope of a histogram constructed by Ln N and Ln L (where L is the length dimension of each square box). The equation previously presented is based on an image where the largest box is 300 pixels.

After the numbers of branch points and end points are found and the fractal dimension is calculated, Block 115 shows that these parameters are compared with predefined levels. The predefined levels may be found by examining large numbers of known angiogeneic images or by using the linear models described above. If the parameters exceed the predefined levels, the image is designated as demonstrating angiogenesis. Block 117 shows that the results may be displayed to a user or analyst.

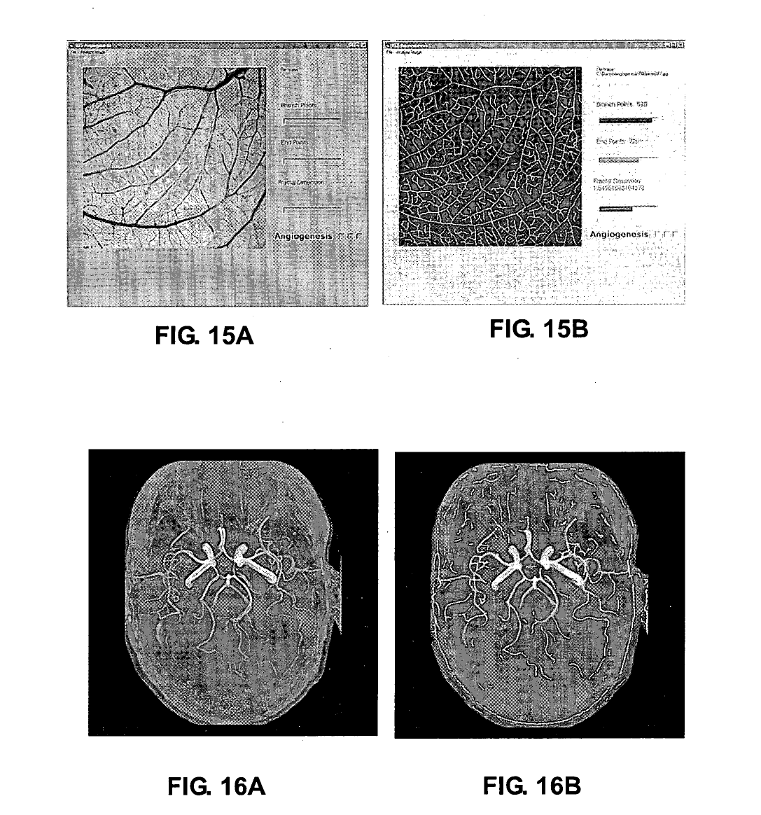

An embodiment of the present invention may be provided by a software program in which user control over the program and results of the program are provided by a Graphical User Interface (GUI). FIGS. 15A and 15B show the GUI provided by an embodiment of the present invention. The File menu option allows a user to load any single image into the image window. The user then presses the “Analyze Image” button, and the program detects the blood vessels, and calculates the corresponding branch points, end points, and fractal dimension. The angiogenesis threshold level (lower right corner of screen image) has already been predetermined for each of these measures. If a feature is over the threshold, the angiogenesis level increases one white square. The results of the levels of angiogenesis and related parameters, including branch points, end-points, and fractal dimensions can be shown in near real-time. FIG. 15A displays an image of the original blood vessels. After the “Analyze Image” button is activated, the program calculates the branch points, end points, and fractal dimension on the detected vessels. FIG. 15B shows the result of line detection. The white squares shown in FIG. 15B indicate the Angiogenesis level automatically, preferably in real time, based on thresholds for branch point counts (>_{—}250), end point counts (>_{—}400), and fractal dimension (>_{—}1.6). The threshold levels for all three critical parameters may be determined by statisticalquantative and statistical analysis. One square will be highlighted if only one parameter meets the threshold criteria. All three squares will be highlighted if all three parameters meet their threshold criteria.

As indicated above, the threshold levels for the determination of the angiogenesis level may be determined by statisticalquantative and statistical analysis. The statistical analysis may comprise the use of a neural network learning process or other adaptive computational method, where a large sample size of images may be analyzed for the determination of the optimum thresholds to be used. The sample size of images may also take into account different situations, e.g., images of breast tissue versus images of brain tissue, to allow for the determination of different thresholds for these different situations. Still other methods may be also used for determining the thresholds. One such method may be the interpretation of statistical charts of the parameters where the charted statistics indicate appropriate thresholds. See, for example, FIGS. 11A-11C, 12A-12C, and 13A-13C above, where charts of the parameters for the various days of the CAM images may be interpreted to indicate appropriate threshold levels for the angiogenesis determination.

Another embodiment of the present invention uses the GUI depicted in FIGS. 15A and 15B and provides additional capabilities for scaling the image to be analyzed and allows an operator to enhance images. Another embodiment of the present invention uses the GUI to allow thresholds to be selected for a Gaussian filter used within a line detection algorithm. Other controls in the GUI may be used to set other parameters in the line detection algorithm and for finding vessels and performing angiogenesis analysis.

Embodiments of the present invention may be used to determine angiogenesis in various situations. For example, brain MRAs may be analyzed in accordance with embodiments of the present invention. FIG. 16A shows an original brain MRA and FIG. 16B shows the same MRA after performing line detection in accordance with embodiments of the present invention. However, some artifacts may arise in the analysis of brain MRAs. These artifacts include:

1. Skull Edge. Lines occur from the boundary of the skull and dark surroundings from the contrast of the gray skull on the black background (white vessels on gray matter).

2. Noise. Bones and different shades in brain tissue are detected by the line detector.

3. Missing Blood Vessels. Not all of the blood vessels display the same color, so it may be difficult to select the right threshold values to extract all the vessels and none of the noise.

4. Incorrect Large Vessel Detection. The line detector may detect several lines within a large vessel network.

Techniques that may be used to address the artifacts that arise with the analysis of brain MRA images are discussed below. For example, if all of the MRA images have a pitch black (grayscale value=0), then a simple threshold will find the background. This can then be used to find the skull boundary and then the location of the detected blood vessels can be checked with respect to the skull boundary. To save computing time, every 5^{th} pixel of the border may be selected. It is recognized that other imaging technologies may be used.

To correct the errors occurring due to the extraction of incorrect blood vessels, a histogram equalization of the image may be performed. This computing technique is generally used to enhance contrast and correct for lighting differences in image processing. This may provide for the enhancement of all of the blood vessels in the image. However, this technique may also brighten the areas around the skull. Hence, the image processing technique should only extract the light areas that contain blood vessels.

Two different scales of the image, full size and half size, may be used for line detection. This would allow for the correction of artifacts generated within the larger vessels. The two extraction results can then be merged into one.

The techniques described above may provide for the removal of skull edge interference and the reduction of noise; the display of previously missing vessels: and the representation of large vessels with only a single line. These techniques also provide that almost all of the vessels are connected to each other. Hence, the additional processing techniques may effectively remove and correct the artifacts that were apparent in images that are not processed using such techniques.

However, more lines may be shown in the brain tissue because of the false positive display of fibrous connective structures as vessels. To solve this problem, a dilation and erosion technique may be used to connect vessels that are close to each other. In this technique, the lines of the detected vessel grow and join if a small gap exists between the vessels. The erosion process (along with a “skeleton” subroutine) is used to shrink the vessels. “Pruning” and “connected regions” methods are used to limit the unwanted noise. Pruning removes any lines less than a certain threshold (e.g., 40 pixels). The “connected regions” method is utilized to remove any line segments that were not connected to another. The pruning and connected regions should eliminate almost all of the false positive errors.

An embodiment of the present invention that may be used to accurately discern blood vessels and detect angiogenesis in brain MRA images may use the following steps:

1. Find the skull border.

2. Enhance the contrast.

3. Employ Gaussian line detection on the full image.

4. Employ Gaussian line detection on the reduced (by 50%) image.

5. Combine the small and the large vessels.

6. Join any discontinuities resulting from dilation and erosion.

7. Eliminate noise, vessels less than a few pixels large, and vessels near the skull border.

8. Analyze the remaining blood vessels by counting branch points and end points, and calculating the fractal dimension.

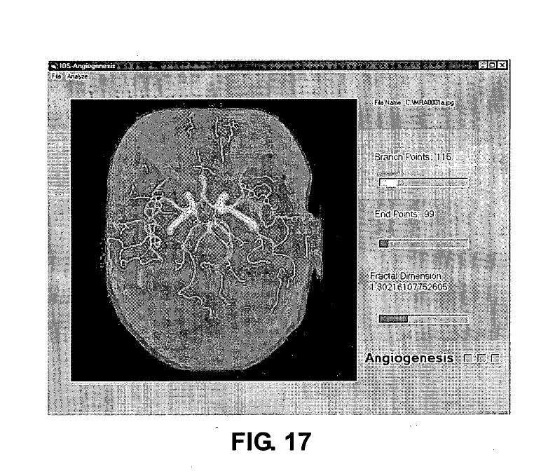

This information may then be displayed to the operator using a simple, user-friendly graphical user interface. FIG. 17 shows an exemplary GUI display for the analysis of a brain MRA image. The analytical result of a human brain MRA shown in FIG. 17 indicates that there are 115 branch points, 99 end points, and the calculated fractal dimension is 1.3. All three parameters are smaller than the pre-established thresholds, so none of the white squares are highlighted, indicating that no angiogenesis was found on this MRA. This is consistent with the diagnosis by an experienced neurological surgeon that it is an MRA of a normal adult subject.

Preferably, the GUI is designed to include the ability to change the image processing parameters (such as Gaussian line detectors, amount of dilation/erosion, etc.). This allows for greater flexibility in processing and analyzing the images, particularly those MRAs containing various levels of angiogenesis, such as those associated with the growth of brain gliomas with various grades of malignancy. Classification software may also be included in the GUI to allow a user to input training image data, perform data fitting (using both linear and non-linear models), display the results, and perform final testing.

Embodiments of the present invention provide a user-friendly, analytical imaging method that can be installed on any personal computer or laptop computer. With such a method and a GUI according to preferred embodiments of the present invention only a few simple commands are required to analyze a large number of images. This will facilitate its use in the battlefield, and in field hospitals, emergency rooms, or clinics. The GUI is preferably designed so that it can be easily used by an operator untrained in cytological techniques.

Embodiments of the present invention are not limited to the analysis of angiogenesis based on two-dimensional images. Other embodiments may operate on three dimensional images that depict blood vessel networks. In an exemplary embodiment, a three dimensional image may be first processed to provide a binarized image in which a one value for a pixel represents the presence of a blood vessel and a zero value represents the absence of a blood vessel. The techniques described above may then be used to process the binarized image to determine the number of branch points, end points, and fractal dimension for the image. These statistical quantative measures may then be compared against thresholds to determine the presence of angiogenesis, although it is likely that the thresholds for three dimensional images will differ from the thresholds for two dimensional images.

The description above describes embodiments of the invention in relation to the determination of angiogenesis, but embodiments of the present invention may be used to provide a statistical quantative measure or measures related to vascularization depicted in an image or a series of images. In accordance with some embodiments of the present invention, statistical quantative measures of vascularization in images from subjects with known conditions (or from subjects who eventually exhibit known conditions) may be made. These statistical quantative measures may then be collected in a database or databases and further analyzed to determine appropriate thresholds or other statistical data for the determination of a correlation between the statistical quantative measures and the known condition. The statistical quantative measures from images from a subject with an unknown condition can then be compared against the database to assist in the determination of whether the subject is likely to have any of the known conditions collected in the database.

In accordance with the vascularization determination described immediately above, an exemplary embodiment of the present invention may provide a determination of the amount of blockage or damage in a vascular structure. The statistical quantative measures from the images from one subject could be compared with the statistical quantative measures from images with subjects with known amounts of damage or blockage. The comparison would then provide some insight into the likely damage or blockage within the examined individual. Other embodiments of the present invention could provide similar analysis for tumors, diseases, etc., for which there is a relationship between vascularization and the type of tumor, disease, etc. In each case, a database may be used to set the statistical standards for whether the type of tumor, disease, etc. is present and the statistical quantative measures from an image or series of images (possibly over time) from a subject are compared with the statistical standards to determine if the subject is likely to have a particular condition.

The foregoing Detailed Description of exemplary and preferred embodiments is presented for purposes of illustration and disclosure in accordance with the requirements of the law. It is not intended to be exhaustive nor to limit the invention to the precise form or forms described, but only to enable others skilled in the art to understand how the invention may be suited for a particular use or implementation. The possibility of modifications and variations will be apparent to practitioners skilled in the art. No limitation is intended by the description of exemplary embodiments which may have included tolerances, feature dimensions, specific operating conditions, engineering specifications, or the like, and which may vary between implementations or with changes to the state of the art, and no limitation should be implied therefrom. This disclosure has been made with respect to the current state of the art, but also contemplates advancements and that adaptations in the future may take into consideration of those advancements, namely in accordance with the then current state of the art. It is intended that the scope of the invention be defined by the Claims as written and equivalents as applicable. Reference to a claim element in the singular is not intended to mean “one and only one” unless explicitly so stated. Moreover, no element, component, nor method or process step in this disclosure is intended to be dedicated to the public regardless of whether the element, component, or step is explicitly recited in the Claims. No claim element herein is to be construed under the provisions of 35 U.S.C. Sec. 112, sixth paragraph, unless the element is expressly recited using the phrase “means for . . . ” and no method or process step herein is to be construed under those provisions unless the step, or steps, are expressly recited using the phrase “comprising step(s) for . . . ”

What is claimed is:

1. A method for determining an extent of vascularization comprising:
generating a digitaldigitalized representation of blood vessels in a selected area;
calculating one or more statistical quantative measures for the blood vessels in the selected area based on the digitaldigitalized representation of the blood vessels; and
comparing the one or more statistical quantative measures to corresponding statistical standards to determine an extent of vascularization.

2. The method as claimed in claim 1, wherein generating a digitaldigitalized representation of blood vessels in a selected area comprises:
converting an image of the blood vessels to a plurality of line segments, wherein only blood vessels in the image having a thickness in excess of a selected thickness are converted to line segments;
connecting neighboring line segments and eliminating gaps between line segments to generate a skeleton representing the blood vessels.
providing parameters related to the skeleton as the digitaldigitalized representation of the blood vessels.

3. The method as claimed in claim 1, wherein the one or more statistical quantative measures are selected from a group consisting of the density of end points contained in the digitaldigitalized representation of the blood vessels, the density of branch points contained in the digitaldigitalized representation of the blood vessels, and the fractal dimension of the digitaldigitalized representation of the blood vessels.

4. The method as claimed in claim 1, wherein the extent of vascularization comprises a determination of the presence or absence of angiogenesis.

5. The method as claimed in claim 4, wherein comparing the one or more statistical quantative measures to corresponding statistical standards comprises determining whether each one of the one or more statistical quantative measures exceeds a corresponding threshold value.

6. The method as claimed in claim 1, wherein the digitaldigitalized representation of blood vessels is a digitaldigitalized representation of blood vessels from a particular subject and the extent of vascularization is used to determine the likelihood of a presence of a particular condition in the subject.

7. The method as claimed in claim 6 further comprising:
generating the corresponding statistical standards, wherein generating the corresponding statistical standards comprises:
calculating the one or more statistical quantative measures for a plurality of digitaldigitalized representations of blood vessels from subjects with known conditions and/or from subjects who eventually exhibit known conditions to provide one or more baseline statistical quantative measures for each digitaldigitalized representation in the plurality of digitaldigitalized representations; and
processing the one or more baseline statistical quantative measures for each digitaldigitalized representation in the plurality of digitaldigitalized representations to determine relationships between the one or more baseline statistical quantative measures and the known conditions; and
calculating the corresponding statistical standards from the relationships between the one or more baseline statistical quantative measures for each digitaldigitalized representation in the plurality of digitaldigitalized representations and the known conditions.

8. The method as claimed in claim 1, wherein the digitaldigitalized representation of the blood vessels in a selected area comprises a series of images of the selected area captured over time and converted to a digital format.

9. The method as claimed in claim 1, wherein the digitaldigitalized representation of the blood vessels comprises one or more two dimensional images of the selected area and/or one or more three dimensional images of the selected area.

10. The method as claimed in claim 1, wherein a user selects the selected area by operating a Graphical User Interface.

11. A computer-readable medium having computer-executable instructions for performing the method as claimed in claim 1.

12. A system for determining the extent of vascularization in a subject comprising:
an input device operable to receive one or more images of blood vessels in the subject;
an output device; and
a processor programmed to:
generate a digitaldigitalized representation of the blood vessels in a selected area based on the one or more images of blood vessels in the subject;
calculate one or more statistical quantative measures for the blood vessels in the selected area based on the digitaldigitalized representation of the blood vessels;
compare the one or more statistical quantative measures to corresponding statistical standards to determine an extent of vascularization; and
send results based on the comparison of the one or more statistical quantative measures to corresponding statistical standards to the output device.

13. The system as claimed in claim 12, wherein the processor generates a digitaldigitalized representation of blood vessels by:
converting at least one image of the blood vessels to a plurality of line segments, wherein only blood vessels in the image having a thickness in excess of a selected thickness are converted to line segments;
connecting neighboring line segments and eliminating gaps between line segments to generate a skeleton representing the blood vessels.
providing parameters related to the skeleton as the digitaldigitalized representation of the blood vessels.

14. The system as claimed in claim 12, wherein the one or more statistical quantative measures are selected from a group consisting of the density of end points contained in the digitaldigitalized representation of the blood vessels, the density of branch points contained in the digitaldigitalized representation of the blood vessels, and the fractal dimension of the digitaldigitalized representation of the blood vessels.

15. The system as claimed in claim 12, wherein the processor is further programmed to determine the presence or absence of angiogenesis based on the extent of vascularization.

16. The system as claimed in claim 15, wherein the system further comprises a database storing a corresponding threshold value for each one of the one or more statistical quantative measures and the processor compares the one or more statistical quantative measures to corresponding statistical standards by determining whether each one of the one or more statistical quantative measures exceeds the corresponding threshold value.

17. The system as claimed in claim 12, wherein processor is further programmed to process the extent of vascularization to determine the likelihood of a presence of a particular condition in the subject.

18. The system as claimed in claim 17 further comprising a database of the corresponding statistical standards, wherein the database is created by:
calculating the one or more statistical quantative measures for a plurality of digitaldigitalized representations of blood vessels from subjects with known conditions and/or from subjects who eventually exhibit known conditions to provide one or more baseline statistical quantative measures for each digitaldigitalized representation in the plurality of digitaldigitalized representations; and
processing the one or more baseline statistical quantative measures for each digitaldigitalized representation in the plurality of digitaldigitalized representations to determine relationships between the one or more baseline statistical quantative measures and the known conditions; and
calculating the corresponding statistical standards from the relationships between the one or more baseline statistical quantative measures for each digitaldigitalized representation in the plurality of digitaldigitalized representations and the known conditions.

19. The system as claimed in claim 12, wherein the one or more images of blood vessels comprises a series of images of a selected area generated at different times.

20. The system as claimed in claim 12, wherein the one or more images of blood vessels in the subject comprise one or more two dimensional images and/or one or more three dimensional images and/or three dimensional angiograms.

21. The system as claimed in claim 12, wherein the system further comprises a Graphical User Interface operable to allow a user to control the images received by the input device and/or to allow the user to select an area in the digitaldigitalized representation of the blood vessels and wherein the Graphical User Interface is further operable to display the results.

22. A method for determining the presence and state of angiogenesis comprising:
receiving an image of a blood vessel network;
extracting an area of interest in the received image;
converting the area of interest in the received image to a plurality of line segments, wherein only blood vessels in the area of interest having a thickness in excess of a selected thickness are converted to line segments;
connecting neighboring line segments and eliminating gaps between line segments to generate a skeleton representing the blood vessels.
calculating a fractal dimension for the skeleton;
comparing the fractal dimension to a fractal dimension threshold value; and
generating a result indicating a presence of angiogenesis if the fractal dimension exceeds the fractal dimension threshold value.

23. The method as claimed in claim 22, further comprising:
calculating a density of branch points in the skeleton;
comparing the density of branch points to a branch point threshold value; and
generating a result indicating a presence of angiogenesis if the density of branch points exceeds the branch point threshold value.

24. The method as claimed in claim 22, further comprising:
calculating a density of end points in the skeleton;
comparing the density of end points to an end point threshold value; and
generating a result indicating a presence of angiogenesis if the density of end points exceeds the end point threshold value.

25. The method as claimed in claim 22, further comprising eliminating line segments closer together than selected distance from the plurality of line segments.

26. The method as claimed in claim 22, further comprising identifying parallel line segments related to sides of at least some of the blood vessels in the received image and converting the parallel line segments to single line segments.

27. The method as claimed in claim 22, wherein the image of the blood vessel network is enhanced before the image is converted to the plurality of line segments.

28. The method as claimed in claim 22, further comprising eliminating line segments having lengths shorter than a selected length from the plurality of line segments.

29. The method as claimed in claim 22, further comprising eliminating line segments having lengths shorter than a selected length from the plurality of line segments.

30. The method as claimed in claim 22, wherein extracting an area of interest in the received image comprises having a user operate a Graphical User Interface to select the area of interest and the result indicating a presence of angiogenesis is displayed by the Graphical User Interface.

31. The method as claimed in claim 4, wherein comparing the one or more quantative measures to corresponding statistical standards comprises determining whether any finction of the one or more quantative measures exceeds a selected threshold value.
